(19) United States
(12) Patent Application Publication
(10) Pub. No.: US 2018/0098587 A1
(43) Pub. Date: Apr. 12, 2018

(54) Control Enhancement for Articles Worn by or Covering a User

(71) Applicant: Jeffrey Wade Moore, Severna Park, MD (US)

(72) Inventor: Jeffrey Wade Moore, Severna Park, MD (US)

(21) Appl. No.: 15/288,142

(22) Filed: Oct. 7, 2016

(51) Int. Cl. A41D 31/00; A41B 1/08; A41D 1/04; A41D 29/00; A41D 1/06; A43B 23/02; F41H 1/02; A62B 17/00; A61F 13/04; A61F 5/01

(52) U.S. Cl. CPC A41D 31/0011; A41B 1/08; A41D 1/04; A41D 29/00; A41D 1/06; A41B 2400/80; F41H 1/02; A62B 17/006; A61F 13/041; A61F 5/0118; A41D 2400/80; A43B 23/0205

(57) ABSTRACT

The invention is the situation of control enhancement on articles worn by or covering a user. Control enhancement is comprised of friction or grip enhancing materials, shapes, coatings or surfaces situated on articles worn on or covering the arms, legs, head, feet, body and some articles warn on the hands of users to improve the ability of users to grasp, carry, control, influence and deflect objects such as balls, tools, weapons and equipment and to improve the user's control over the user's orientation, position and overall situation. Articles worn by or covering a user (e.g. garments, gear, uniforms, equipment, devices, sleeves, bands, pants, suits, pads) can make it difficult to maintain control of objects or for users to maintain control over their circumstances (e.g. slipping down a smooth surface). Control enhancements situated optimally as a plurality, especially in opposing locations, aid the user and may be purposefully arranged to enhance the utility and appearance of an article worn by or covering a user.

### CROSS-REFERENCE TO RELATED APPLICATIONS

This application claims the benefit of cross-reference Provisional Patent Application No. 62/248,410 filed on Oct. 30, 2015. The title of the invention which is the subject of aforementioned Provisional Patent Application number is Friction Surface for Garments, the disclosure of which is incorporated herein by reference.

### STATEMENT REGARDING FEDERALLY SPONSORED RESEARCH OR DEVELOPMENT (IF APPLICABLE)

The subject of this application and this application are not by or for any federal, state or local government and not sponsored research or development by any federal, state or local government. The section is not applicable.

### REFERENCE TO SEQUENCE LISTING, A TABLE, OR A COMPUTER PROGRAM LISTING COMPACT DISC APPENDIX (IF APPLICABLE)

Not Applicable

### BACKGROUND OF THE INVENTION

The invention generally relates to control enhancement for articles worn by or covering a user. Articles worn by or covering users include (include means including, but not limited to) clothing, suits, uniforms, gear, equipment, and devices. Such articles include, but are not limited to, clothing of all types, sports, military and other uniforms, protective or decorative articles, tactical articles, shoes, boots, socks, shirts, tops, pants, bottoms, jackets, hats, emergency response garb, suits, chemical resistant suits, fire retardant suits, survival suits, weather gear, foul weather gear, coats, shields, casts, braces, supports, coverings, articles worn on or covering the extremities including the hand, wrist, forearm, head, face, legs, shins, ankles, feet, fingers, thumbs, toes and other locations and similar items that are worn by or covering a user. The invention improves the utility of the articles by reducing slip and enhancing user control over objects and or user's circumstance. Slip is reduced by increasing the amount of friction or adhesion between the control enhancement situated on the article and an object over which a user desires to have or increase control or influence, especially through the exertion of pressure, such as balls, tools, weapons, persons, living things, and inanimate objects. It also increases user's control over user's situation, including circumstances, position and location, when the user is wearing articles improved with the invention such as when the user is in contact with surfaces, especially where pressure is exerted in a circumstance, for example pressing hands, fingers, thumbs, feet, toes, legs, back, torso and or other areas against smooth, semi-smooth and rough surfaces such as glass, rock, metal, wood, plastic and other materials. Currently, articles allow or cause users to drop objects or lose a degree of control over objects or the article causes interference or otherwise does not adequately aid in control or influence over objects or the article fails to adequately help the user to control the user's situation. Situation of control enhancement for articles worn by or covering a user into or onto such articles improves user control and influence over objects and the user's situation, persons, living things and inanimate objects.

Some attempts to address the problem of control or grasp have not sufficiently addressed the needs of users or the industry because the attempts were bulky, stiff or binding or simply did not provide for adequate location, coverage or inclusion onto or into the article or compromised the intrinsic characteristics and performance of the article (e.g. loss of stretch or breathability). The attempts restrict movement or interfere with control over a user's situation, persons, living things, inanimate objects and the like by being bulky, stiff, or inadequately located, for example. Other attempts did not provide for adequate retention of article characteristics, flexibility, breathability, stretch, coverage, versatility, location, orientation or aesthetics to adequately aid the user in enhancing control, for example over objects, in a range of uses without detracting from the use of the article. The subject invention solves the problem without loss of article characteristics, utility and or versatility. Therefore, the subject invention is useful not only in football, soccer and other athletics endeavors, but also it is useful in a variety of work, rescue, firefighting, emergency response, scientific, educational, and research endeavors, and enhances the utility of, for example, chemical resistant suits, para-military, law enforcement, and military and other applications and endeavors where the user's ability to maintain control over objects, other persons, and the user's situation, while maintaining user freedom of movement and reasonable retention of article characteristics are important. Moreover, in particular the subject invention includes control enhancement in the form of pictures, messages, words, shapes, logos, camouflage, visibility, and the like through a plurality of control enhancements added to the article to form pictures, messages, words, shapes, logos and the like and coatings to be situated onto or into articles to improve user control. The control enhancements are situated into or onto articles, or on material later affixed or added to an article, as a plurality, for example of small dots, hemispheres or shapes, parallelograms, and may be arranged to form pictures, shapes, designs, messages, letters, words, logos or a coating that improves user control and better allows the article to retain its basic performance characteristics (e.g. stretch, compression, tightness, looseness and breathability).

### BRIEF SUMMARY OF THE INVENTION

The invention is the situation of control enhancement on articles worn by or covering a user. Control enhancement is comprised of friction, grasp, stick or grip enhancing surfaces situated on articles worn on or covering any part of users including all parts of a user. Parts of a user may include, but not be limited to arms, legs, ankles, shins, knees, buttocks, head, feet, toes, body, abdomen, back, hands, fingers, thumbs, wrists, and neck for example. Parts of a user may also include, but not be limited to skin, shell, coverings, wings, wheels, and other parts, for example. The control enhancement is situated to improve the ability of users to grasp, carry, control, influence and deflect objects such as balls, tools, weapons and equipment and to improve the user's control over the user's situation. Users include athletes, sports participants, firefighters, military persons, emergency responders, environmental cleanup personnel, among others and users may be robots or animals, for example, animals trained to carry out tasks in which control enhancement is useful. Users may be living beings or inanimate objects such as robots or drones, for example. Situation of control enhancement into or onto articles worn by or covering users solves the problem of too little control over objects and the user's situation by increasing a user's ability to grasp, influence and control objects, person, inanimate objects and to enhance the user's control over user's orientation, location, position or situation.

### BRIEF DESCRIPTION OF THE SEVERAL VIEWS OF THE DRAWINGS

There are eight drawings included with the application. The drawings are labeled as figures and descriptions of each figure are provided below.

FIG. 1 shows a particular version of control enhancement situated on articles including clothing and garments such as a shirt or top.

FIG. 2 shows a particular version of control enhancement situated on articles including a uniform, such as one including a jersey or top and pants or bottoms.

FIG. 3 shows a particular version of control enhancement situated on articles including gear such as compression sleeve.

FIG. 4 shows a particular version of control enhancement situated on articles such as the tops of shoes and boots.

FIG. 5 shows a particular version of control enhancement situated on articles worn on or covering the hand (including fingers and thumb) such as a cast or cast cover.

FIG. 6 shows a particular version of control enhancement situated on articles including devices such as a brace.

FIG. 7 shows a particular version of control enhancement situated on articles including equipment such as a tactical or bullet-proof vest.

FIG. 8 shows a particular version of control enhancement situated on articles including gear such as a chemical-resistant suit.

### DETAILED DESCRIPTION OF THE INVENTION

The invention is the situation of control enhancement for articles worn by or covering a user. It is comprised of friction or grip enhancing materials situated on articles worn on or covering the arms, legs, head, feet, toes, body and some articles warn on the hands, fingers and thumbs of users to improve the ability of users to grasp, carry, control, influence and deflect objects such as balls, tools, weapons and equipment and to improve the user's control over the user's situation when pressure is applied by the user.

More specifically, the invention involves the situation of control enhancement onto or into articles including clothing, suits, uniforms, gear, equipment and devices worn or covering a user, comprising;
a. said control enhancements being a coating or multiple coatings, situated as a plurality of smaller surfaces or a combination of same on articles such that the control enhancement improves user control over objects with pressure applied by the user, whether or not user intentionally applies pressure;
b. said control enhancements being a coating or multiple coatings, situated as a plurality of smaller surfaces or a combination of same on articles such that the control enhancement improves user control over user's circumstances, position, situation, location and or orientation, for example, with pressure applied (passively, actively, intentionally or unintentionally) by the user;
c. said control enhancements being camouflage, colored, natural, black, white, or any color, clear, translucent, opaque, or any combination of appearances;
d. said control enhancements situated in a pattern or otherwise in designs, shapes, forms, messages, logos, numbers, letters, words, before or after location on an article;
e. said control enhancements being rubber, rubbery material, sticky, tacky, glue-like material, resin, rough, smooth, dull, shiny, reflective, invisible, elastic, leather, hide, synthetic hide, synthetic material, liquid, gel, gel-filled, hard, flexible, soft, hollow, filled, semi-solid and or solid material;
f. said control enhancements being one or more of the above materials in any combination;
g. said control enhancements being situated into or onto the outer surface of articles;
h. said control enhancements being added into or onto material that is in turn situated into or onto the article whether by adhesion, coating, stitching, thermal treatment, or otherwise (e.g. uniform numbers, logos, patches, etc.);
i. said control enhancements being situated by a method including, but not limited to, pouring, spreading, smearing, dripping, painting, spraying, dabbing, dapping, squeezing, pressing, heating, cooling, thermally treating, sewing, stitching, gluing, sticking, adhering, weaving, clipping, welding, tacking, fluxing, soldering, tying, combining, forming, enveloping, including, attaching, affixing, physical or chemical attachment to or on and or inclusion in an article;
j. said control enhancements being of a size, location, arrangement, and composition suitable to be a plurality of surfaces whether small or large that work or function individually or in concert to promote user's influence or control on or over any object or thing that user contacts, holds, catches, blocks, deflects, grips, grasps, clutches, clings to, squeezes or attempts to exert some amount of control or influence over or upon or by which user attempts to control user's balance, position, orientation, concealment, announcement, movement or location, for example;
k. said control enhancements being such to preserve the characteristics of the article to a reasonable extent;
l. such control enhancements being such that the article on which they are situated performs reasonably as the article would otherwise perform especially in terms of stretch, flexibility or inflexibility, breathability, vapor flow, water flow, air flow, to the degree possible after including control enhancement, for example;
m. said control enhancements being made a part of the article or made a part of a material that in turn is made a part of article on which they are a part in the form or arrangement of a coating or a plurality of solid or hollow drips, bubbles, spheres, cubes, squares, cones, dots, hemispheres, specks, bumps, hair-like, indentations, shapes, dimples, lines, blocks, patterns, letters, numbers, numerals, words, messages, pictures, regular, irregular and random arrangements, and figures and combinations of such forms and arrangements, for example;
n. said articles being clothing, suits, uniforms, gear, foul weather gear or suits, vests, medical devices, protective and other equipment, and devices by locating the control enhancement in the initial creation of the article and/or by later location on the article.

The situation of control enhancement on an article as recited above, further comprising one or more said control enhancements situated on a garment on the sleeves of the garment between the shoulder and wrist, which garments are worn as shirts, insulating shirts, undershirts, tops, outerwear, jerseys, uniform tops, shells, jackets, compression shirts, and other garments and articles warn on the upper body, in particular, with control enhancements situated along the arms of garments from above the elbow to the wrist on the side of the sleeve that is adjacent to the body of the garment.

The situation of control enhancement on an article as recited above, further comprising one or more control enhancements situated on a garment on the front of such garments, such that the control enhancements cover or extend over the front of the article from the side adjacent to the inside arm across the front portion of the garment between the top or neckline and bottom of the garment, to the other side and adjacent to the inside of the opposite arm.

The situation of control enhancement on an article as recited above, further comprising one or more control enhancements situated along the thighs, waist, crotch areas on the front of garments, which are warn as pants, shorts, bottoms, uniform bottoms, pants, trousers, shells, outerwear and other garments on the lower body, such that the control enhancements cover or extend over the front and sides of each leg and front and sided of crotch and waist area.

The situation of control enhancement on an article as recited above, further comprising one or more said control enhancements situated on the article comprising and worn as wrist bands, wrist pads, forearm bands, forearm pads, forearm sleeves, play holders, elbow sleeves, elbow pads, shoulder pads, compression sleeves, compression bands, protective sleeves, bands or pads, straps, bands, pads, tubes casts, cast covers, braces, shells, wraps, or similar devices worn on or covering the arms between the shoulder and wrist, or the body at the abdomen, waist or chest, or the legs at the thigh.

The situation of control enhancement on an article as recited above, further comprising one or more said control enhancements situated on the article and worn as armor, camouflage, visibility, invisibility, protection, covering, thermal protection or cooling, or comfort on the body of the user.

The situation of control enhancement on an article as recited above, further comprising one or more control enhancements in any combination of arrangements on any article of clothing, uniform, gear, device or equipment worn by or covering a user.

Articles worn by or covering users such as garments (e.g. shirts, tops, pants, bottoms, sleeves, jerseys, uniforms, jackets, gear, bands, equipment, pads, devices, straps, braces, emergency response garb, etc.) sometimes fail to aid in, or make it difficult to maintain, control of objects or the user's situation. Articles worn by or covering users enhance user control over objects and the user's situation when control enhancement (e.g. rubberized and or sticky drops, or a similar material, thin coatings and shapes) are situated onto or into the outer surface of the garment in the form of a plurality of dots, hemispheres, drips, strips, lines, cross hatches, dimples, raised surfaces, directional raised “teeth” as seen in snake's teeth, or other patterns or designs, which are included in the article's original production or added at a later time, for example in an after-market way (e.g. sew-on, iron-on or stick on, etc.) or added on a material that is then added to the article (e.g. numbers or letters containing control enhancement that are added to a jersey).

For example, a football player wearing long sleeves has a tendency to lose control over a football when attempting to catch or carry the football. A player wearing sleeves or a uniform containing control enhancement for articles optimally situated between the shoulder and writs, for example, onto or into the sleeve and/or body of the uniform (or multiple other locations) catch and retain control over a football better than a player whose sleeves or uniform does not have control enhancement situated optimally (e.g. a plurality or thin coating) on the article. A soccer player for example benefits from control enhancement into or onto shoes, cleats, boots, socks and jerseys. Similarly, football punters and kickers or soccer players benefit from control enhancement on the tops of shoes and on socks, and a soldier benefits from control enhancement in or on the front and or back of uniforms, tactical suits, tactical vests, sporting vests (e.g. fishing or hunting), bullet-proof vests, special operation gear, and other equipment, gear, and devices. A soldier has better control of held or carried objects and the soldier's own situation (e.g. climbing on rock, wood, metal or other materials or objects) when wearing articles containing control enhancement. Rescuers, firefighters, medical personnel, emergency responders or environmental cleanup personnel, for example, wearing protective suits containing control enhancement have improved control over objects and their situations and can work safer. In the case of hazardous environments, control enhancement should be compatible with the hazard, environment, use, needs and situation of the user.

The invention reduces the ease with which a ball, for example, in contact with garments slips out of control. The control enhancement is situated as a plurality of shapes or coatings on articles worn by or covering users. For example, control enhancement situated on long sleeves between the wrist and shoulder and in an opposing location on the body of a garment in the chest, waist, abdomen between the top and bottom of the article aids the user to control held or carried objects when user applies pressure for the purpose of exerting control over the object. Likewise, control enhancement on pants or shorts between the top and bottom of the article, for example in the areas of the thighs and lap aids the user's control. The control enhancement may be clear, translucent, colored or patterned to accomplish a desired aesthetic or need for uniforms, practice garments and other articles and may be situated in the shape of pictures, letters or symbols for marketing, communication, decoration and function (e.g. camouflage, team logos, inspirational messages, numbers, etc.).

The control enhancement is situated on articles worn by or covering users to improve user grasp and control of objects and situation. The best mode is to situate control enhancements in opposing locations on an article, or on different articles worn together or separately such that control enhancement(s) on the articles are in opposing locations, as a plurality of raised parallelograms (i.e. opposite angles equal and adjacent angles not equal) up to 0.5-inch base length more or less by up to 0.25-inch height more or less and preferably raised from less than 0.1 millimeter up to 0.5 millimeters in elevation above the surface of the article and each parallelogram separated by approximately 0.125-inches more or less on all sides, or thin coatings or, alternatively, dots or hemispheres (up to 0.125-inch diameter more or less and preferably raised as described above), reasonably evenly distributed and separated by approximately the diameter of a dot or hemisphere more or less (edge to nearest edge) or, alternatively, raised cones set vertically or on angles and arranged in directional rows or “teeth” such as is seen in snake's teeth, (up to 0.125-inch diameter at base more or less and preferably raised as described above), reasonably evenly distributed and separated by approximately the diameter of the base of a cone more or less (edge to nearest edge) such that the surfaces work or function together to improve user grasp and control of an object, for example a football, and user control over the user's situation (e.g. orientation, position, or location). This scenario entails situating control enhancement on the arms from wrist to shoulder (or near the arm pit at the top of the sleeve) facing the body of the shirt or top and on the abdomen of the shirt or top (if the shirt or top contains an abdomen section), or an article of uniform placed over the shirt or top, facing the forearm so that a football, for example will be in contact with the control enhancement over as much area of the football as is practical. Control enhancement on one article may be used in combination control enhancement on another article (e.g. a long sleeve undershirt containing control enhancement situated on the arms may be used in combination with a football jersey containing control enhancement situated on the abdomen to aid user in controlling a football when pressure is applied by user).

The situation of control enhancement into or onto articles worn by or covering users solves the problem of lack control over objects and the user's situation by increasing a user's ability to grasp, influence and control objects, person, inanimate objects and the user's orientation, location, position or situation. These improvements promote more successful, competent, fun, exciting, effective and safe performance, respectively, depending on the task performed.

Control enhancement may be situated on the fronts and sides of articles in most instances, but not always, and may be situated over the entire garment. For example, the control enhancement should be situated on the back of certain articles, or on the back in addition to other locations, where a user relies on the back of the article to maintain control, for example, over the user's position, orientation or location in a confined space such as a vertical shaft where user may apply pressure from user's back to the walls of the shaft.

A user may be a person, an animal inanimate thing, such as a robot.

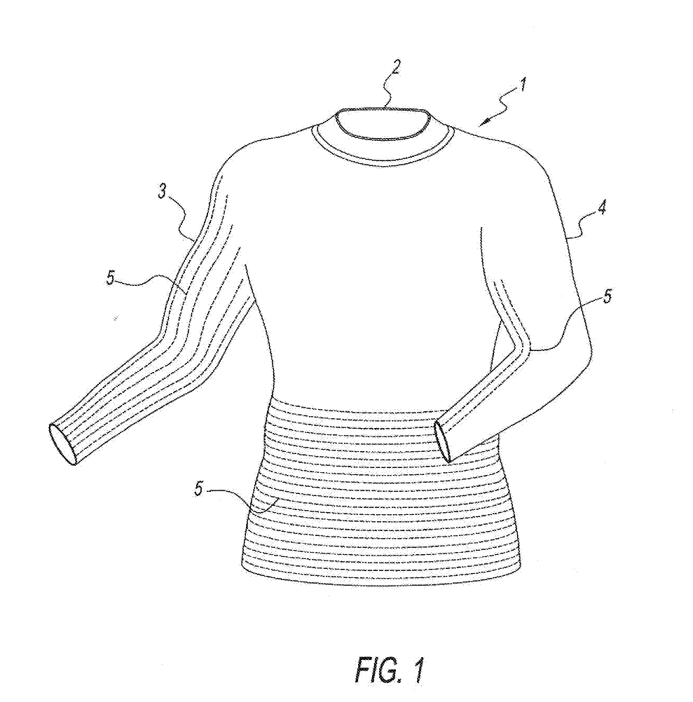

FIG. 1 shows a particular version of control enhancement for an article worn by or covering a user. In this particular embodiment the control enhancement is shown on a shirt 1, facing front forward and having a crew or round shirt neck 2, with the right sleeve 3 extended right and left sleeve 4 bent partially across body. The right sleeve 3 is shown extended to aid in showing the control enhancement 5 situated on the right sleeve 3. Control enhancement 5 is situated on the parts of the sleeves that are closest to the body of the shirt 1. Control enhancement 5 is also situated on the left sleeve 4, but it partially hidden from view in FIG. 1 in this example. Control enhancement 5 is shown as dashed lines in the example, but shapes may vary. The length or area covered by control enhancement 5 is optional as shown. Control enhancement 5 may be applied over the entirety of the article (not shown). Particularly suitable control enhancement includes camouflage, colored, natural, black, white, or any color, clear, translucent, opaque rubber, rubbery material, sticky, tacky, glue-like material, resin, rough, smooth, dull, shiny, reflective, invisible, elastic, leather, hide, synthetic hide, synthetic material, liquid, gel, gel-filled, hard, flexible, soft, hollow, filled, semi-solid and or solid material (not shown). Particularly suitable locations and configurations for control enhancement 5 may include the locations on an article that frequently come into contact with an object over which the user desires to have some control or influence or a surface that affects the user's situation. Control enhancement 5 is comprised of a plurality of smaller surfaces or a single, continuous coating or unit. Articles are not limited to shirts and may include pants, tops, bottoms, jackets, socks, hats, gloves, and other garments.

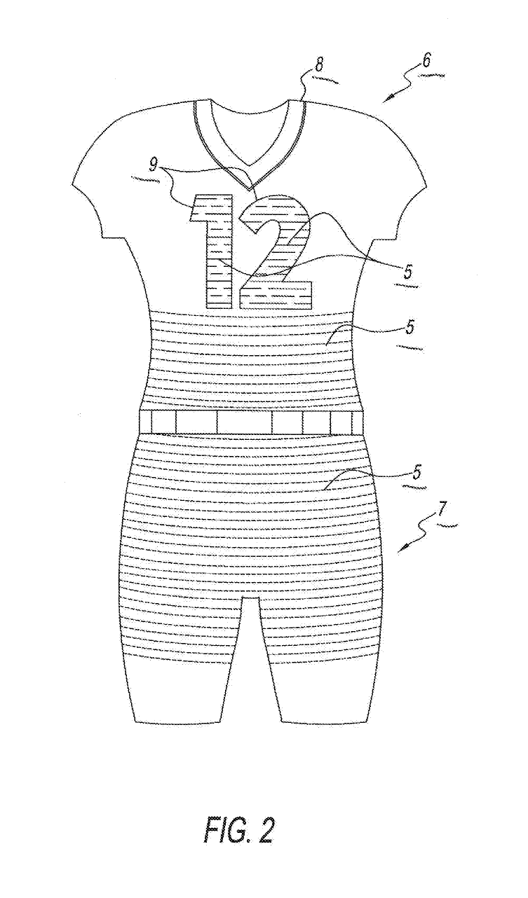

FIG. 2 shows a particular version of a control enhancement for an article. In this particular embodiment the control enhancement for an article is shown with a uniform, more specifically a football uniform for example, comprised of a jersey 6 and pants 7, facing front forward and the jersey 6 having a “v” jersey neck 8. Control enhancement 5 is situated on the part of the body of the jersey 6 of the uniform between the jersey neck 8 and bottom of the jersey. In this particular example, control enhancement 5 is also shown on the numerals 9 attached to or on the front of the jersey 6. Control enhancement 5 is also situated on the pants 7 portion of the uniform on the front between the top and bottom of the pants 7. The length or area covered by control enhancement 5 is optional as shown. Control enhancement 5 may be applied over the entirety of the uniform jersey 6 (not shown) and pants 7 (not shown). Control enhancement 5 is shown as dashed lines in the example, but shapes may vary. Particularly suitable control enhancement include those described for FIG. 1 camouflage, colored, natural, black, white, or any color, clear, translucent, opaque rubber, rubbery material, sticky, tacky, glue-like material, resin, rough, smooth, dull, shiny, reflective, invisible, elastic, leather, hide, synthetic hide, synthetic material, liquid, gel, gel-filled, hard, flexible, soft, hollow, filled, semi-solid and or solid material (not shown). Particularly suitable locations and configurations for control enhancement 5 may include those descried for FIG. 1 the locations on an article that frequently come into contact with an object over which the user desires to have some control or influence or a surface that affects the user's situation. Control enhancement 5 is comprised of a plurality of smaller surfaces or a single, continuous coating or unit. Articles are not limited to uniforms. Other articles may include but not be limited to shorts, kilts, skirts, socks, jump suits, protective clothing, undergarments and other articles including outerwear (not shown).

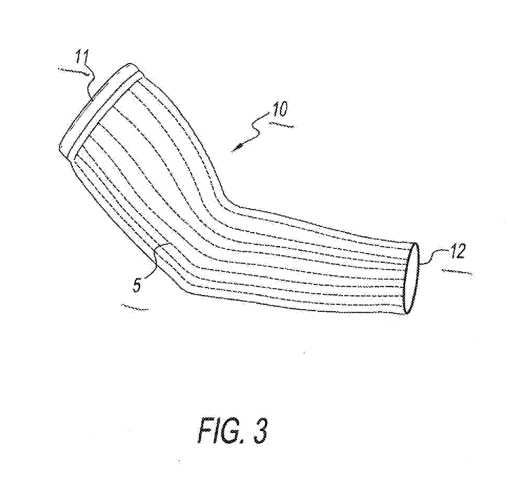

FIG. 3 shows a particular version of a control enhancement for an article. In this particular embodiment the control enhancement is shown with a type of equipment or gear, more specifically a compression sleeve 10 as an example, but may be situated on other equipment or gear such as wrist bands (not shown), forearm pads (not shown), play holders (not shown) and straps and bands (not shown). The compression sleeve 10 is shown with the part of the sleeve that would be nearest the trunk of the body when worn facing forward for ease of viewing. The top 11 of the article is the part nearest the shoulder when worn and the bottom 12 of the article is the part nearest the wrist when worn. Control enhancement 5 is situated on the outer surface of the part of the compression sleeve 10 that would be closest to the trunk of the body (not shown) of the user when worn. Control enhancement 5 is shown as dashed lines in the example, but shapes may vary. The length or area covered by control enhancement 5 is optional as shown. Control enhancement 5 may be applied over the entirety of the article (not shown). Particularly suitable control enhancement includes camouflage, colored, natural, black, white, or any color, clear, translucent, opaque rubber, rubbery material, sticky, tacky, glue-like material, resin, rough, smooth, dull, shiny, reflective, invisible, elastic, leather, hide, synthetic hide, synthetic material, liquid, gel, gel-filled, hard, flexible, soft, hollow, filled, semi-solid and or solid material (not shown). Particularly suitable locations and configurations for control enhancement 5 may include the locations on an article that frequently come into contact with an object over which the user desires to have some control or influence or a surface that affects the user's situation. Control enhancement 5 is comprised of a plurality of smaller surfaces or a single, continuous coating or unit. Articles are not limited to compression sleeve equipment or gear. Other equipment or gear may include but not be limited to wrist bands, wrist pads, play-holders, straps, forearm sleeves, forearm pads, elbow sleeves, elbow pads, and other straps, bands, pads, tubes, wraps, tapes, adhesive surfaces, casts, cast covers, braces, shells, socks, and any other equipment or gear worn by or covering a user.

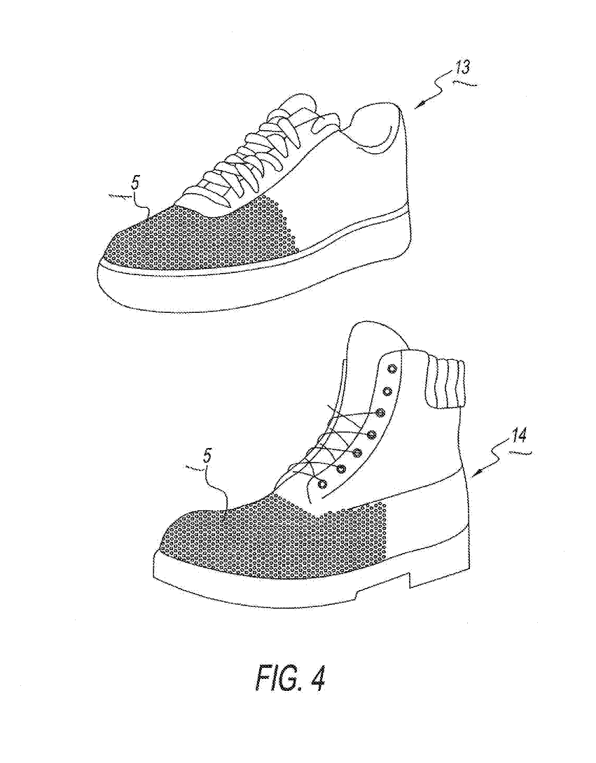

FIG. 4 shows a particular version of a control enhancement for articles. In this particular embodiment the control enhancement 5 is shown on a left shoe 13 and a left boot 14 as examples. Control enhancement 5 may be situated in both shoes (not shown) and both boots (not shown), or for special applications such as a football kicker or punter, control enhancement 5 may be situated on the left or right shoe or boot, as desired. The length and area covered by control enhancement 5 is optional as shown. Control enhancement 5 is shown as small polygons or hemispheres in the example, but shapes may vary. The length and area covered by control enhancement 5 is optional as shown. Control enhancement 5 may be applied over the entirety of the article (not shown). Particularly suitable control enhancement includes camouflage, colored, natural, black, white, or any color, clear, translucent, opaque rubber, rubbery material, sticky, tacky, glue-like material, resin, rough, smooth, dull, shiny, reflective, invisible, elastic, leather, hide, synthetic hide, synthetic material, liquid, gel, gel-filled, hard, flexible, soft, hollow, filled, semi-solid and or solid material (not shown). Particularly suitable locations and configurations for control enhancement 5 may include the locations on an article that frequently come into contact with an object over which the user desires to have some control or influence or a surface that affects the user's situation. Control enhancement 5 is comprised of a plurality of smaller surfaces or a single, continuous coating or unit. Articles are not limited to shoes or boots and may include but not be limited to cleats, skates, prosthetics, sandals, flip flops, clogs, and other footwear and devices coming into contact with the ground and other surfaces, materials or objects.

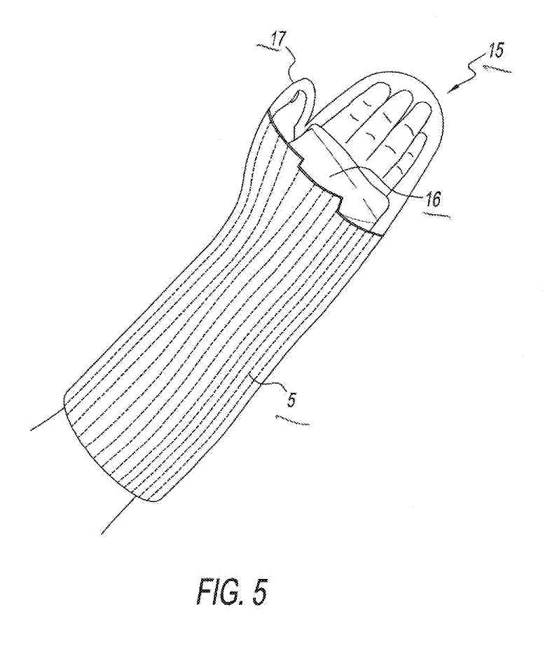

FIG. 5 shows a particular version of a control enhancement for articles. In this particular embodiment the control enhancement is shown with a device, more specifically a cast 15 in the example, with palm 16 facing forward and thumb 17 up. This particular example is shown in cut-away view partially revealing the palm side of the hand and wrap or cast on hand and arm when worn. The length and area covered by control enhancement 5 is optional as shown. Control enhancement 5 is shown as dashed lines in the example, but shapes may vary. The length of the control enhancement 5 is optional as shown. Control enhancement 5 may be applied over the entirety of the article (not shown) or on covers used to cover or pad casts (not shown). Particularly suitable control enhancement includes camouflage, colored, natural, black, white, or any color, clear, translucent, opaque rubber, rubbery material, sticky, tacky, glue-like material, resin, rough, smooth, dull, shiny, reflective, invisible, elastic, leather, hide, synthetic hide, synthetic material, liquid, gel, gel-filled, hard, flexible, soft, hollow, filled, semi-solid and or solid material (not shown). Particularly suitable locations and configurations for control enhancement 5 may include the locations on an article that frequently come into contact with an object over which the user desires to have some control or influence or a surface that affects the user's situation. Control enhancement 5 is comprised of a plurality of smaller surfaces or a single, continuous coating or unit. Articles are not limited to cast devices or cost covers. Other devices may include but not be limited to prosthetic devices, medical devices, straps or braces and similar devices (not shown).

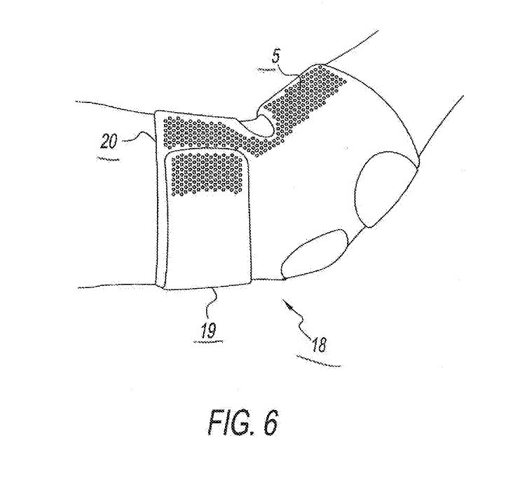

FIG. 6 shows a particular version of control enhancement for articles. In this particular embodiment the control enhancement is shown with brace device as an example. The brace is an elbow brace 18 shown with strap 19 or straps (not shown) and a metal, plastic or other material forming the structure (not shown) of the brace 18. The brace is shown with the top 20 being the part nearest the shoulder and the part nearest the body when worn facing forward in the example. Control enhancement 5 is situated on the structural part of the brace 18 (if exposed, on the covering if covered) and the strap 19 or straps (not shown). Control enhancement 5 is shown as small polygons or hemispheres in the example, but shapes may vary. The length and area covered by control enhancement 5 are optional as shown. Control enhancement 5 may be applied over the entirety of the article (not shown) or a portion of the article as shown. Particularly suitable control enhancement includes camouflage, colored, natural, black, white, or any color, clear, translucent, opaque rubber, rubbery material, sticky, tacky, glue-like material, resin, rough, smooth, dull, shiny, reflective, invisible, elastic, leather, hide, synthetic hide, synthetic material, liquid, gel, gel-filled, hard, flexible, soft, hollow, filled, semi-solid and or solid material (not shown). Particularly suitable locations and configurations for control enhancement 5 may include the locations on an article that frequently come into contact with an object over which the user desires to have some control or influence or a surface that affects the user's situation. Control enhancement 5 is to be comprised of a plurality of smaller surfaces or a single, continuous coating or unit. Articles are not limited to brace devices. Other devices may include but not be limited to prosthetic devices, medical devices, casts, cast covers, straps or other braces and similar devices (not shown).

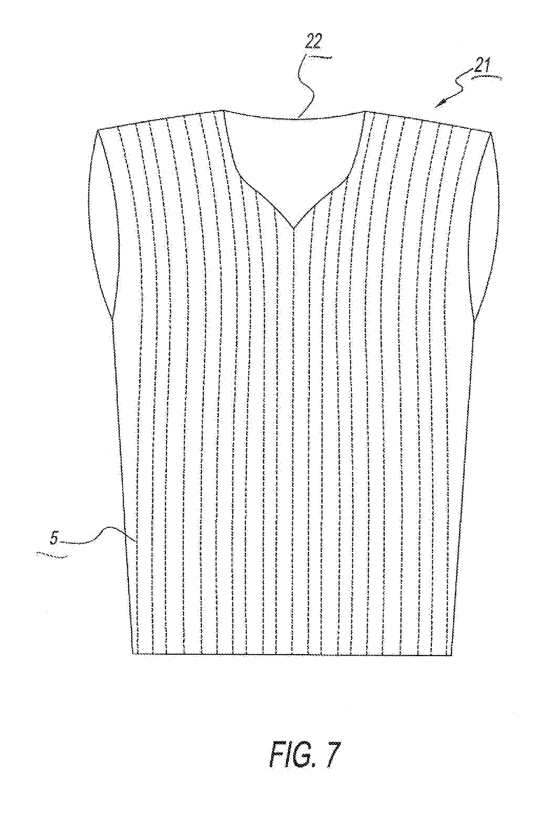

FIG. 7 shows a particular version of control enhancement for an article of equipment worn by or covering a user. In this particular embodiment the control enhancement with a tactical or bullet-proof vest 21 in the example, with the vest neck 22 up and facing front forward. The equipment may be other forms of shields, pads or other equipment (not shown). Control enhancement 5 is situated over the entire front of the tactical or bullet-proof vest 21 in the example. Control enhancement 5 is shown as dashed lines in the example, but shapes may vary. The length and area covered by control enhancement 5 are optional as shown. Control enhancement 5 may be applied over the entirety of the article (not shown). Particularly suitable control enhancement includes camouflage, colored, natural, black, white, or any color, clear, translucent, opaque rubber, rubbery material, sticky, tacky, glue-like material, resin, rough, smooth, dull, shiny, reflective, invisible, elastic, leather, hide, synthetic hide, synthetic material, liquid, gel, gel-filled, hard, flexible, soft, hollow, filled, semi-solid and or solid material (not shown). Particularly suitable locations and configurations for control enhancement 5 may include the locations on an article that frequently come into contact with an object over which the user desires to have some control or influence or a surface that affects the user's situation. Control enhancement 5 is to be comprised of a plurality of smaller surfaces or a single, continuous coating or unit. Articles are not limited to tactical or bullet-proof vest gear or equipment. Other gear or equipment may include, but not be limited to, shields, packs, pads, explosion resistant suits, sporting vests, tactical articles, and other articles worn by or covering users that may contact objects, materials or surfaces.

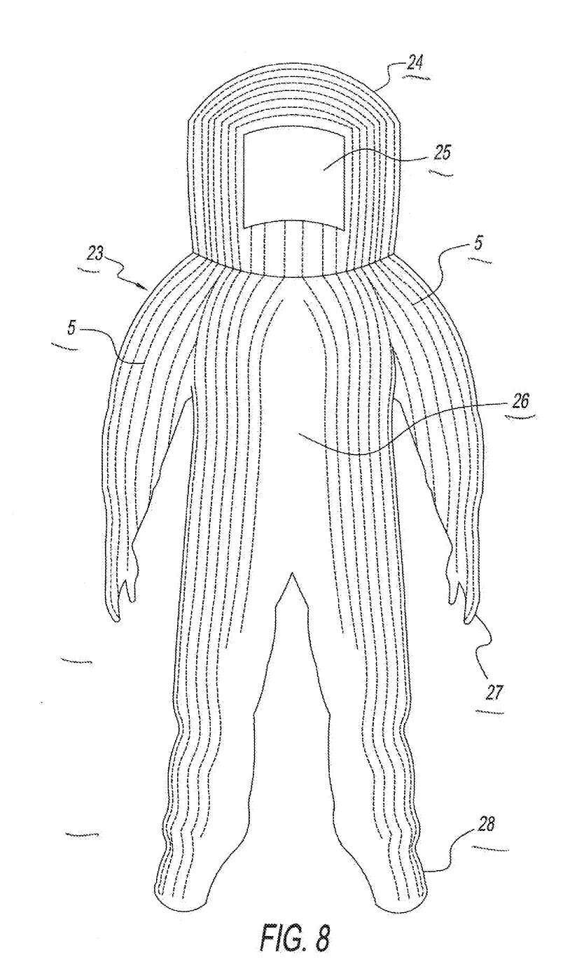

FIG. 8 shows a particular version of control enhancement for an article worn by or covering a user. In this particular embodiment the control enhancement is shown on a chemical resistant suit 23, facing front forward and as having a hood 24 and face shield 25. Control enhancement 5 is situated on the body 26 and hood 24 of the chemical resistant suit 23 and on the area covering the arms, hands 27, fingers, thumbs, legs, toes and feet 28, including the soles of the feet. Control enhancement 5 is shown as a plurality of dashed lines in the example, but shapes may vary. The length and area covered by control enhancement 5 is optional as shown. Control enhancement 5 may be applied over all (not shown) or a portion of the article, but location on the face shield 25 is not recommended. If control enhancement is situated on the face shield it must not interfere or otherwise impede user's sight, movement, health or safety (e.g. it must be of a location, material, color, clarity, etc. to ensure and promote safe and effective use). Particularly suitable control enhancement includes camouflage, colored, natural, black, white, or any color, clear, translucent, opaque rubber, rubbery material, sticky, tacky, glue-like material, resin, rough, smooth, dull, shiny, reflective, invisible, elastic, leather, hide, synthetic hide, synthetic material, liquid, gel, gel-filled, hard, flexible, soft, hollow, filled, semi-solid and or solid material (not shown). Resistant, for example, chemical and or fire resistant, materials are recommended for this version and any other application involving protection of a user from environmental hazards. Particularly suitable locations for control enhancement 5 may include the locations on an article that frequently come into contact with an object over which the user desires to have some control or influence or a surface that affects the user's situation. Control enhancement 5 is to be comprised of a plurality of smaller surfaces or a single, continuous coating or unit. Articles are not limited to chemical resistant suits, gear or equipment. Other gear or equipment may include, but not be limited to, gear or equipment used to enter potentially hazardous environments such as confined spaces, lagoons, low-oxygen environments, fires, fully or partially submerged locations, and other articles and forms of outerwear worn by or covering a user that may contact objects, materials or surfaces and other devices used with articles worn by or covering a user.

What is claimed is:

1. The situation on an article of at least one control enhancement, comprising;
said control enhancement being of material(s) that reduce slip between its surface and another surface when user pressure is applied; and
said control enhancement being, situated on an article such that, in addition to reducing slip between its surface and another surface when user pressure is applied, the article and material comprising the article on which the control enhancement is situated perform reasonably as the article and material comprising the article would otherwise perform.

2. The situation on an article of at least one control enhancement of claim 1, further comprising articles worn by a user or users (hereinafter the terms user and users may be used interchangeably);

3. The situation on an article of at least one control enhancement of claim I, further comprising articles covering all or part of a user or users;

4-13. (canceled)

14. The situation on an article of at least one control enhancement of claim 1, further comprising said control enhancement of a size, location, arrangement, orientation and composition that promotes user's control and or influence over objects that user contacts.

15. The situation on an article of at least one control enhancement of claim 1, further comprising said control enhancement of a size, shape material, location, arrangement, orientation and composition that reduces slip between the article on which the control enhancement is situated and any other thing contacting the control enhancement or which the control enhancement contacts including the ground, surfaces, materials, objects, users, living beings and or inanimate objects.

16. The situation on an article of at least one control enhancement of claim 1, further comprising said control enhancement of a size, shape material, location, arrangement, orientation and composition that promotes user's influence or control over any other thing coming into contact with the control enhancement including the ground, surfaces, materials, objects, users, living beings and or inanimate objects.

17. The situation on an article of at least one control enhancement of claim 1, further comprising said control enhancement of a size, shape, material, location, arrangement, orientation and composition that promotes user's control over user's situation.

18. The situation on an article of at least one control enhancement of claim 1, further comprising said control enhancement of a size, shape, material, location, arrangement, orientation and composition that promotes user's control over user's orientation.

19. The situation on an article of at least one control enhancement of claim 1, further comprising said control enhancement of a size, shape, material, location, arrangement, orientation and composition that promotes user's control over user's position.

20. The situation on an article of at least one control enhancement of claim 1, further comprising said control enhancement of a size, shape, material, location, arrangement, orientation and composition that promotes user's control over user's location.

21-118. (canceled)
